(19) United States
(12) Patent Application Publication
(10) Pub. No.: US 2015/0032106 A1
(43) Pub. Date: Jan. 29, 2015

(54) VESSEL CORSET FOR USE WITH ELECTROSURGICAL FORCEPS

(71) Applicant: Covidien LP, Mansfield, MA (US)

(72) Inventor: THOMAS M. RACHLIN, SAN DIEGO, CA (US)

(73) Assignee: COVIDIEN LP, Mansfield, MA (US)

(21) Appl. No.: 14/282,738

(22) Filed: May 20, 2014

Related U.S. Application Data
(60) Provisional application No. 61/858,028, filed on Jul. 24, 2013.

(51) Int. Cl. A61B 18/14

(52) U.S. Cl. CPC A61B 18/14
USPC 606/51

(57) ABSTRACT

A system for treating tissue is provided. The system includes a forceps having opposing jaw members at a distal end thereof configured to treat tissue. A corset is configured to be wrapped around tissue and stretched to reduce an overall cross section of tissue and allow the jaw members to grasp the tissue for surgical treatment thereof

### CROSS REFERENCE TO RELATED APPLICATION

The present application claims the benefit of and priority to U.S. Provisional Application Ser. No. 61/858,028, filed on Jul. 24, 2013, the entire contents of which are incorporated herein by reference.

### BACKGROUND

1. Technical Field

The present disclosure relates to a vessel corset configured for use with electrosurgical forceps. More particularly, the present disclosure relates to a vessel corset configured for placing tissue in a reduced cross-sectional condition for grasping and subsequent treatment thereof.

2. Description of Related Art

Electrosurgical forceps are known in the art. Both the scissor and laparoscopic type of forceps may be configured to couple to one or more suitable energy sources and may include a pair of opposing jaw members that are configured to grasp and electrosurgically treat tissue (e.g., blood vessels). The scissor and laparoscopic type of forceps may have a maximum threshold for a width or cross-section of vessels that the forceps may be configured to electrosurgically treat. That is, the forceps may have one or more components (e.g., jaw stop) that are configured to limit how far apart the jaw members can be moved away from one another. As a result thereof, a surgeon may have to grasp and electrosurgically treat a vessel at different locations along a treatment zone of the vessel to effectively treat the vessel, e.g., seal the tissue. As can be appreciated, this may increase surgery time, materials utilized in the sealing procedure and may limit a practical use of the forceps.

### SUMMARY

A vessel corset configured for placing tissue in a reduced cross-sectional condition suitable for grasping and subsequent treatment thereof may prove useful in the surgical arena.

Embodiments of the present disclosure are described in detail with reference to the drawing figures wherein like reference numerals identify similar or identical elements. As used herein, the term “distal” refers to the portion that is being described which is further from a user, while the term “proximal” refers to the portion that is being described which is closer to a user.

An aspect of the present disclosure provides a system for treating tissue. The system includes a forceps that is configured to treat tissue. The forceps may be an electrosurgical forceps of the open or closed type. A corset is configured to be wrapped around tissue and stretched to reduce the overall cross section of tissue and allow the jaw members to grasp the tissue for surgical treatment thereof.

The corset may be formed from a material that allows the corset, after being stretched, to maintain the tissue in the reduced cross-sectional condition. For example, the corset may be formed from a polymeric material.

The corset may include a plurality of apertures along a length thereof that are configured to allow electrosurgical energy to pass from the jaw members, through the corset and to tissue. The corset may be stretchable along an axis that is parallel to a longitudinal axis defined through the tissue. The corset may include a low tack adhesive layer that is configured to couple opposing ends of the corset to one another to allow a user to secure the corset around the tissue and stretch the corset along the longitudinal axis defined to reduce the overall cross section of the tissue.

An aspect of the present disclosure provides a method for electrosurgically treating tissue. Specifically, a deformable corset may be wrapped around tissue of interest. Opposing ends of the corset may then be coupled to one another to secure the corset to the tissue of interest. The corset may be stretched to a deformed condition with a reduced cross section to facilitate grasping the corresponding reduced cross section of tissue with a pair of opposing jaw members. The corset may be grasped with the jaw members. And, electrosurgical energy may be applied to the tissue of interest to electrosurgically treat the tissue of interest.

The corset may be formed from a material that allows the corset, after being stretched, to maintain the tissue in the reduced cross-sectional condition. For example, the corset may be formed from a polymeric material.

The corset may include a plurality of apertures along a length thereof that are configured to allow electrosurgical energy to pass from the jaw members, through the corset and to tissue. The corset may be stretchable along an axis that is parallel to a longitudinal axis defined through the tissue. The corset may include a low tack adhesive layer that is configured couple opposing ends of the corset to one another to allow a user to secure the corset around the tissue and stretch the corset along the longitudinal defined through the tissue.

An electrosurgical endoscopic forceps may be utilized for the electrosurgical forceps. Alternatively, an electrosurgical open type forceps may be utilized for the electrosurgical forceps. Further, a knife blade of the electrosurgical forceps may be advanced to sever the electrosurgically treated tissue of interest.

### BRIEF DESCRIPTION OF THE DRAWING

Various embodiments of the present disclosure are described hereinbelow with references to the drawings, wherein:

FIG. 1 is a perspective view of an electrosurgical instrument in accordance with an embodiment of the instant disclosure;

FIG. 2 is a schematic, plan view of a corset configured for use with the electrosurgical forceps shown in FIG. 1;

FIG. 3 is a perspective view of the corset shown in FIG. 2 with the corset wrapped around tissue and in a pre-stretched configuration;

FIG. 4 is a perspective view of the corset shown in FIG. 2 with the corset wrapped around tissue and in a stretched configuration; and

FIG. 5 is a perspective view of an electrosurgical instrument configured for use with the corset shown in FIG. 2.

### DETAILED DESCRIPTION

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

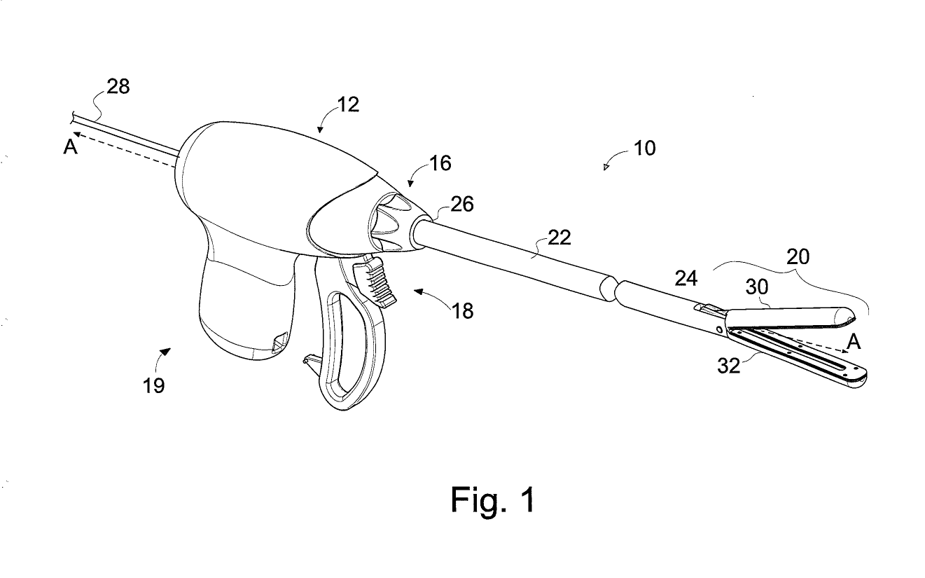

FIG. 1 illustrates an electrosurgical endoscopic forceps 10 (forceps 10) configured for electrosurgically treating tissue. Briefly, forceps 10 includes a housing 12, a handle assembly 14, a rotating assembly 16, a trigger assembly 18 and an end effector assembly 20. A shaft 22 extends from the housing 12 and has a longitudinal axis “A-A” defined therethrough. A distal end 24 of shaft 22 is configured to mechanically engage end effector assembly 20 and a proximal end 26 is configured to mechanically engage housing 12. Forceps 10 also includes an electrosurgical cable 28 that connects forceps 10 to a generator (not shown) or other suitable power source. Forceps 10 may alternatively be configured as a battery-powered instrument. The generator may be configured to provide electrosurgical energy (e.g., RF, microwave, optical, etc.,), thermal energy, ultrasonic energy, and the like to jaw members 30 and 32 of the end effector assembly 20. The forceps 10 includes a knife blade (not explicitly shown) that is advanceable through the jaw members 30, 32 to sever tissue. For a more detailed description of the forceps 10, reference is made to U.S. patent application Ser. No. 13/461,355 filed on May 1, 2012 by Allen IV et al.

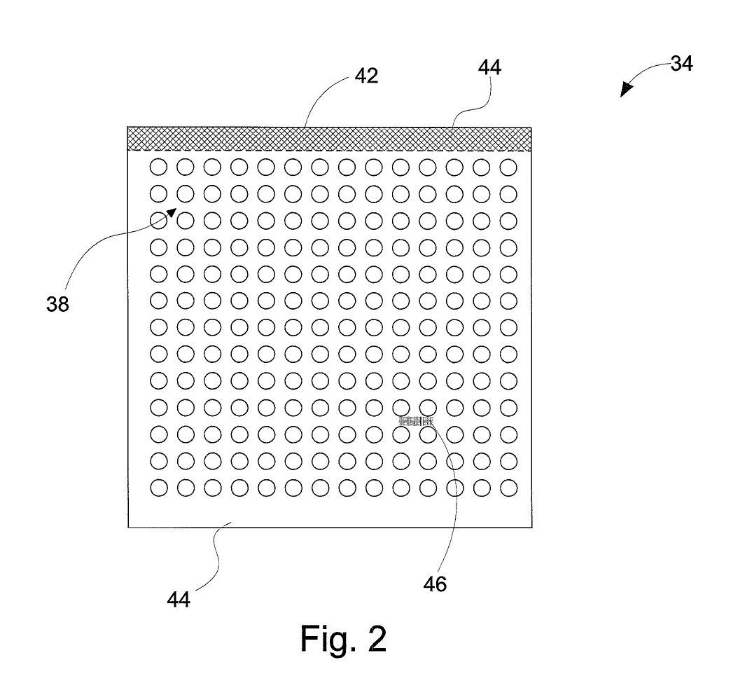

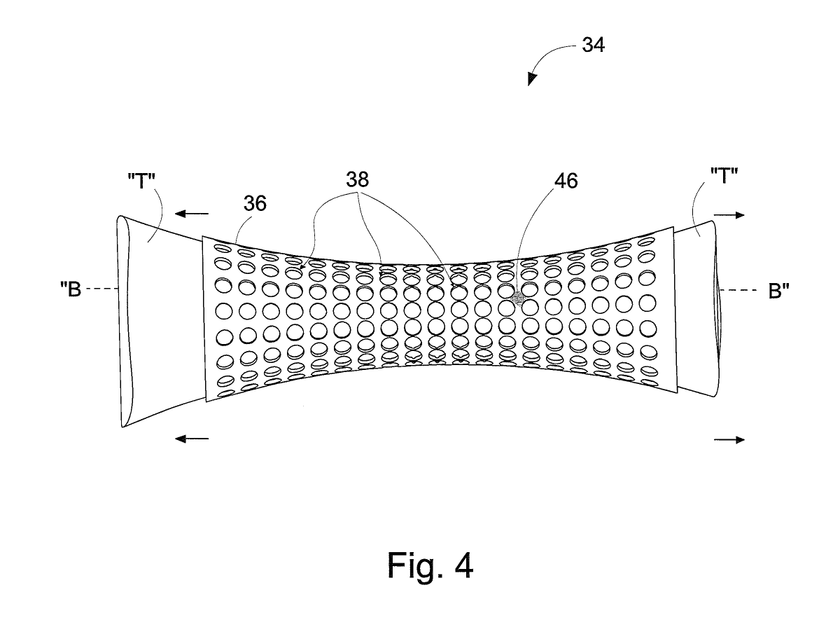

With reference to FIGS. 2-4, a vessel corset 34 (corset 34) configured for use with the forceps 10 is illustrated. The corset 34 may be formed from one or more suitable biocompatible materials. In the illustrated embodiment, for example, corset 34 is formed from a polymer mesh 36 (FIG. 4). The polymer mesh 36 is configured so as to allow the corset 34, after being stretched (or deformed), to maintain the tissue “T” in the reduced cross-sectional condition, as best seen in FIG. 4. That is, the corset 34 maintains the stretched condition once stretched. Corset 34 is biocompatible such that the corset 34 may remain on the tissue during electrosurgical treatment of the tissue (e.g., during a sealing procedure of the tissue) and in a body cavity of a patient after the sealing procedure is completed.

Corset 34 is configured to wrap around vessels which are too large to seal or dissect with traditionally-sized jaw members 30, 32. That is, the jaw members 30, 32 are limited to how far apart they can be moved and may not be able to accommodate large-sized vessels. Corset 34 is configured to be wrapped around tissue “T” and stretched by a user for placing the tissue in a reduced cross-sectional condition suitable for being grasped by traditionally-sized jaw members 30, 32 such that electrosurgical energy may be applied to tissue “T.” In other words, with the tissue in the reduced cross-sectional condition, a user can position the tissue between the jaw members 30, 32 and clamp the jaw members 30, 32 on tissue for subsequent treatment thereof.

The corset 34 may include a plurality of apertures 38 that are disposed along a length thereof in a uniform or non-uniform pattern. The apertures 38 may include any geometrical configuration including without limitation, rectangular, circular (as shown), triangular, oval, etc. The apertures 38 are configured to allow electrosurgical energy to pass from the jaw members 30, 32, through the corset 34 and to tissue “T.”

The corset 34 is stretchable (or deformable) along an axis that is parallel to a longitudinal axis “B-B” defined through the tissue “T” such that the corset 34 compresses the tissue into the reduced cross-sectional condition for tissue treatment.

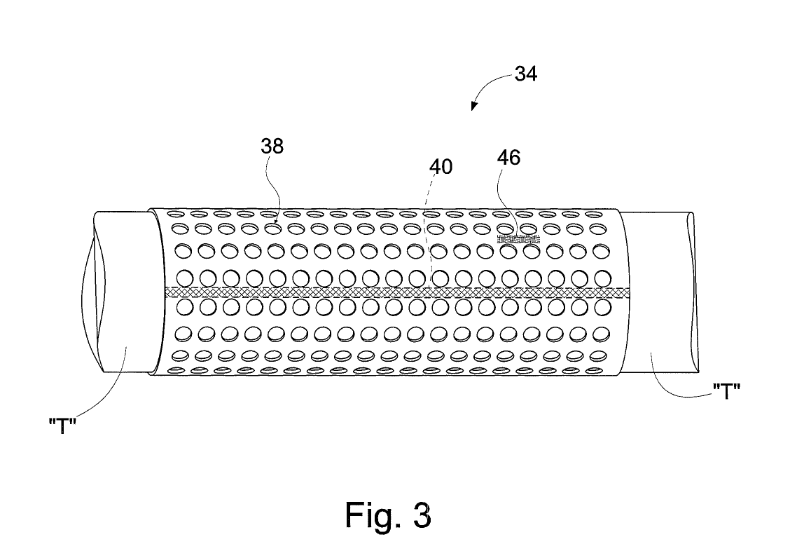

The corset 34 may include a low tack adhesive layer 40 (FIGS. 2-3) configured to allow a user to couple opposing ends 42, 44 of the corset 34 to one another about a vessel. In the illustrated embodiment, for example, the adhesive layer 40 is provided on the opposing end 42 of the corset 34. A user can fold the opposing end 42 over the opposing end 44 to couple the opposing ends 42, 44 to one another. In accordance with the instant disclosure, the corset 34 will include a generally cylindrical configuration when the opposing ends 42, 44 are coupled to one another (FIG. 3). This allows a user to wrap the corset 34 around tissue “T” and secure the corset 34 to the tissue “T” such that the corset 34 can be stretched along the longitudinal axis “B-B” defined through the tissue “T.” Other coupling methods may be utilized to couple the opposing ends to one another.

In use, corset 34 may be shipped with the opposing ends 42, 44 uncoupled to from one another (FIG. 2). The corset 34 may be wrapped around tissue of interest “T.” Opposing ends 42, 44 of the corset 34 may be coupled to one another to secure the corset 34 to the tissue of interest “T” (FIG. 3).

The corset 34 may then be stretched by a user to place the tissue of interest “T” in a reduced cross-sectional condition suitable for being grasped by jaw members 30, 32 of end effector 20 and for applying electrosurgical energy to the tissue of interest “T.” A user may then grasp the corset 34 with the jaw members 30, 34. Thereafter, electrosurgical energy may be applied to the tissue of interest “T” to electrosurgically treat the tissue of interest “T.”

The corset 34 described herein gives a surgeon the ability to seal tissue having large cross-sectional areas with a single grasping of the tissue. As can be appreciated, this may decrease surgery time and materials utilized in the sealing procedure, and may increase a practical use of the forceps 10.

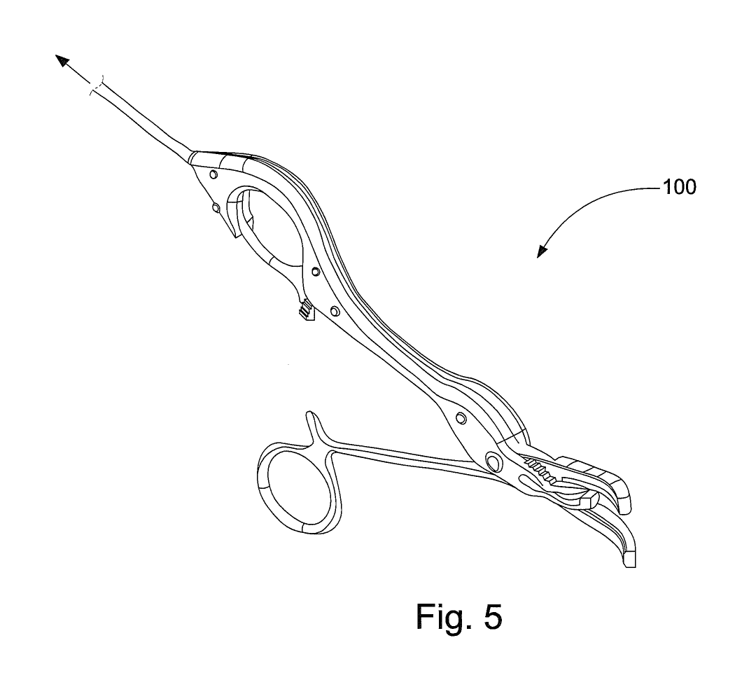

From the foregoing and with reference to the various figure drawings, those skilled in the art will appreciate that certain modifications can also be made to the present disclosure without departing from the scope of the same. For example, while the corset 34 has been shown and described herein as being configured for use with an endoscopic forceps 10, corset 34 may be utilized with an open style type forceps 100 (i.e., scissor type), see FIG. 5.

In embodiments, the corset 34 may be shipped with the opposing ends 42, 44 coupled to one another along a perforated edge (not explicitly shown) that extends along the corset 34. In this embodiment, a user can separate the opposing ends 42, 44 along the perforation and couple the opposing end 42, which includes the adhesive layer 40, to the opposing end 44 in a manner as described above.

In embodiments, the corset 34 may be at least partially coated with or formed from a lubricious material 46. In this embodiment, the lubricious material 46 is configured to reduce sticking between the corset 34 and seal plates (not explicitly shown) of the jaw members 30, 32 during electrosurgical treatment of the tissue “T.”

While the corset 34 has been described herein including a plurality apertures 38 that are uniformly disposed along the corset 34, it is within the purview of the instant disclosure to utilize other aperture 38 patterns or configurations. For example, the apertures 38 may be non-uniformly disposed along the corset 34 to achieve a specific seal pattern across the tissue

While the corset 34 has been described herein as being configured for use with electrosurgical instruments, it is within the purview of the instant disclosure that the corset 34 may be utilized with surgical instruments that are not configured to electrosurgically treat tissue, e.g., graspers, forceps, dissectors, etc. Such instruments may be configured to grasp and/or cut, sever and/or dissect tissue.

The various embodiments disclosed herein may also be configured to work with robotic surgical systems and what is commonly referred to as “Telesurgery”. Such systems employ various robotic elements to assist the surgeon in the operating theatre and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A system for treating tissue, comprising:
a forceps having opposing jaw members at a distal end thereof configured to treat tissue; and
a corset configured to be wrapped around tissue and stretched to reduce an overall cross section of tissue and allow the jaw members to grasp the tissue for surgical treatment thereof.

2. The system according to claim 1, wherein the corset is formed from a material that allows the corset, after being stretched, to maintain the tissue in the reduced cross-sectional condition.

3. The system according to claim 2, wherein the corset is formed from a polymeric material.

4. The system according to claim 1, wherein the forceps is an electrosurgical forceps and the corset includes a plurality of apertures along a length thereof that are configured to allow electrosurgical energy to pass from the jaw members, through the corset and to tissue.

5. The system according to claim 1, wherein the corset is stretchable along an axis that is parallel to a longitudinal axis defined through the tissue.

6. The system according to claim 5, wherein the corset includes a low tack adhesive layer that is configured to couple opposing ends of the corset to one another to allow a user to secure the corset around the tissue and stretch the corset along the longitudinal axis to reduce the overall cross section of the tissue.

7. A method for electrosurgically treating tissue, comprising:
wrapping a deformable corset around tissue of interest;
coupling opposing ends of the corset to one another to secure the corset to the tissue of interest;
stretching the corset to a deformed condition with a reduced cross-section to facilitate grasping the corresponding reduced cross section of the tissue with a pair of opposing jaw members;
grasping the corset with the jaw members; and
applying electrosurgical energy to the tissue of interest to electrosurgically treat the tissue of interest.

8. The method according to claim 7, including forming the corset from a material that allows the corset, after being stretched, to maintain the tissue in the reduced cross-sectional condition.

9. The method according to claim 8, wherein forming includes forming the corset from a polymeric material.

10. The method according to claim 7, including providing a plurality of apertures along a length of the corset to allow electrosurgical energy to pass from the jaw members, through the corset and to the tissue of interest.

11. The method according to claim 7, including stretching the corset along an axis that is parallel to a longitudinal axis defined through the tissue of interest.

12. The method according to claim 7, including providing a low tack adhesive layer along the corset, the low tack adhesive configured to couple the opposing ends of the corset to one another around the tissue of interest.

13. The method according to claim 7, including utilizing an electrosurgical endoscopic forceps for the electrosurgical forceps.

14. The method according to claim 7, including utilizing an electrosurgical open type forceps for the electrosurgical forceps.

15. The method according to claim 7, further including advancing a knife blade of the electrosurgical forceps to sever the electrosurgically treated tissue of interest.
